(19) United States
(12) Patent Application Publication
(10) Pub. No.: US 2017/0182204 A1
(43) Pub. Date: Jun. 29, 2017

(54) A BIOACTIVE TISSUE REPAIRING LAYER

(71) Applicants: Deniz TANYOLAC, Ankara (TR); Yasemin GÜNAYDIN, Ankara (TR)

(72) Inventors: Deniz TANYOLAC, Ankara (TR); Yasemin GÜNAYDIN, Ankara (TR)

(21) Appl. No.: 15/508,383

(22) PCT Filed: Sep. 2, 2015
(86) PCT No.: PCT/TR15/00297

§ 371 Date: Mar. 2, 2017

(30) Foreign Application Priority Data
Sep. 2, 2014 (TR) 2014/10245

(51) Int. Cl. A61L 15/44; A61L 15/58; A61L 15/34; A61L 15/60; A61L 15/20; A61K 36/481; A61L 15/40

(52) U.S. Cl. CPC A61L 15/44; A61K 36/481; A61L 15/58; A61L 15/40; A61L 15/60; A61L 15/20; A61L 15/34; A61L 2300/30; A61L 2300/412

(57) ABSTRACT

The present invention relates to a repairing layer which covers at least a part of wound upon being applied inside or onto a wound and comprises water, ozonized oil, glycerol and astragalus plant extract.

### TECHNICAL FIELD

The present invention relates to a repairing film layer which is used for healing a wound with medium and high exudate; can transform into a gel form by swelling inside or onto a wound in a controlled way; and which is biodegradable, biocompatible, adhesive, transparent and elastic.

### BACKGROUND OF THE INVENTION

Wound dressings are materials which are used for providing protective characteristic against microorganisms by covering wounds, absorbing blood and wound fluid, ensuring wound healing, and applying medical treatment onto wound in some cases. The said materials may be a film layer and the ones being commonly used in the state of the art may be in the form of gel or a textile product wherein the active substance is fed. However, the active substance has an effect on the wound only as a primary dressing in wound dressings used in the form of gel whereas in the event that the wound is covered by a textile product it causes not to be able to observe the healing process of the wound and requires renewing the dressing during recovery. In addition, due to the fact that the said dressings create a pressure on the wound for example bleedings may happen in the wound or the patient may be uncomfortable due to feeling of pressure.

Using of unsuitable dressing may disrupt circulation or may harm newly formed tissue. And this causes wound healing to be delayed. A suitable wound dressing should be applied easily, should not harm the newly formed tissue during removal, should create an optimal humidity environment for wound healing and require less often dressing change interval.

The United States patent document with number US2013337090 discloses plant extracts which are used for treating chronic wounds and burns. In the said documents, it is disclosed that wounds or burns are treated by mixing a plant extract with an oil and it is mentioned that olive oil can be used as oil and astragalus plant is used as plant extract. It is also disclosed in the said document that the composition prepared can be in the form of a wound dressing.

The Chinese patent document with number CN102178632 discloses use of ozonized oil for health-care products. In the invention described in the said document, it is stated that ozonized olive oil is used in toothpastes, bacterial creams or medical dressings due to its antibacterial characteristic.

### SUMMARY OF THE INVENTION

An objective of the present invention is to realize a wound repairing layer wherein there is only astragalus plant extract and ozonized olive oil.

Another objective of the present invention is to realize a wound repairing layer which is in the form of film; is transparent, adhesive, elastic; has oxygen permeability; exhibits high antimicrobial activity.

Another objective of the present invention is to realize a wound repairing layer which provides controlled release of active substances.

Another objective of the present invention is to realize a wound repairing layer whereby healing process can be followed by enabling to observe the wound constantly.

Another objective of the present invention is to realize a biodegradable repairing layer which transforms into a gel form on or inside the wound.

Another objective of the present invention is to realize a wound repairing layer which is herbal and does not comprise chemical substance.

Another objective of the present invention is to realize a wound repairing layer which can be applied to infected wound in any type of stage.

Another objective of the present invention is to realize a wound repairing layer which does not lead to any trauma on skin by being subjected to degradation with body fluid while performing wound healing.

Another objective of the present invention is to realize a wound repairing layer which does not create pressure on wound.

Another objective of the present invention is to realize a wound repairing layer which does not adhere to the wound wherein the part contacting the wound has a hydrophilic structure.

Another objective of the present invention is to realize a wound repairing layer wherein the outer part not contacting the wound is transformed into hydrophobic structure by using a cross-linker.

Another objective of the present invention is to realize a wound repairing layer which can remain on the wound according to wound characteristic and duration of treatment.

### DETAILED DESCRIPTION OF THE INVENTION

A repairing layer which covers at least a part of wound upon being applied inside or onto a wound comprises water, ozonized olive oil, glycerol and astragalus plant extract,

The repairing layer developed by the present invention is composed in an elastic film form which is generated by combining distilled water, preferably ozonized olive oil having high peroxide value in terms of its wound healing characteristic, glycerol and astragalus plant extract; is preferably transparent and can adhere to the region where the wound is present without needing any extra adhesive material (self-adhesive). The said film layer swells in the course of time by interacting with the wound and therefore it realizes controlled release of active substance by transforming into gel form in the region where it is applied. In addition, the layer developed by the present invention prevents transition of microorganisms, can continuously keep the wound moisturized and also ensures controlled release of gas/water vapour transmission to the wound.

In a preferred embodiment of the invention, the repairing layer comprises at least one cross-linker for the purpose of increasing mechanical characteristics according to the type of wound where it will be applied, the place of use and the time how long it will remain on the wound. The said cross-linker is genipin in one embodiment of the invention whereas it is calcium chloride in another embodiment of the invention. Based on the characteristic of the wound where the said cross-linker will be applied, its amount inside the film layer can be increased or reduced and thus the action time of the active substance on the wound, in other words the release time, can be adjusted.

In a preferred embodiment of the invention, the layer comprises ozonized olive oil between 0.5 to 6 (v/v) by percentage.

In a preferred embodiment of the invention, the layer comprises glycerol between 2 to 10 (v/v) by percentage.

In a preferred embodiment of the invention, the layer comprises astragalus plant extract between 0.5 to 3 (w/v) by percentage.

In a preferred embodiment of the invention, the layer is carried out by a method of immersion into a solution comprising cross-linker in the ratio of 5 (w/v) by percentage.

In a preferred embodiment of the invention, peroxide value of ozonized oil is between the range of 1000 to 1500 meg oxygen/kg oil.

In another preferred embodiment of the invention, the layer comprises at least one protection band which covers the layer and can be removed during application.

In another preferred embodiment of the invention, the layer comprises at least one adhesive material which is located on the side to be applied to the wound and ensures adherence in the area where it is applied.

Astragalus plant, Latin name: Astragalus membranaceus, stimulate immune system cells by increasing antibodies, macrophages, T cells and natural killer cell activity and increases interferon production while enhancing blood cell activity due to bioflavonoids, choline, saponin, polysaccharide, astragalan B, calcium, magnesium and potassium that it contains. In addition, it protects the body by fighting against bacteria, fungi and viruses; regulates production of tumor necrosis factor by increasing natural killer cell activity; repairs aging cells; improves immune system; and accelerates wound healing in an efficient way. Extract of astragalus plant which is grown in central, eastern and south-eastern regions of our country by itself and also known as tragacanth is still used in our country for only paper marbling (Ebru) and producing tragacanth doll.

Ozone gas (O₃) is a gas having a very-high oxidation potential. Ozone gas leads to formation of new compounds from olive oil by breaking double bonds existing in Olive oil and/or adding oxygen molecule. These new molecules existing in the product show effect by triggering some biochemical reactions which show effect for formation of new vessel (vascularization) in tissue and tissue regenerating.

When ozonized olive oil is applied to skin, ozone molecules which have not reacted in oil also have an effect on skin directly in addition to newly formed compounds and exhibit a high antimicrobial activity against many bacteria, fungi and viruses generated in the wound by ensuring that intensity of this interaction is increased. Ozonized oil exhibits a substantial antimicrobial activity on microorganisms causing particularly skin infections such as trichophyton, microspum and epidermophyton. It is known in the literature that ozonized sunflower seed oil significantly prevents infections created by mycobacteria, gram-positive and gram-negative bacteria on skin.

Ozone, which is used for closing open wounds occurring in diabetic patients, can also be applied to diabetic patients having no open wounds in order to prevent tissue damage that may occur in the future. Pressure sores (decubitus ulcer) occurring based on lying in bed for a long time, serious wounds occurring in legs (ulcus cruris), skin allergies, psoriasis, persistent acne, neurodermatitis are main application fields of ozone treatment.

Due to the fact that the layer developed by the present invention is a thin film layer, it applies no pressure onto the wound and thus, for example, bleeding of wound due to pressure is avoided and the patient is enabled to have no discomfort.

At the same time, healing period of wound can be watched easily because the film layer has a transparent form. Due to the fact that the film layer transforms into gel form by swelling inside or on the wound, it is not necessary anymore to change the layer and it is ensured that the wound heals fast by means of the ozonized olive oil and astragalus plant extract with high peroxide value that it contains.

It is possible to develop a great variety of embodiments regarding the invention “A Repairing Layer”, it cannot be limited to the examples disclosed herein and it is essentially according to the claims.

1. A repairing layer which covers at toast a part of wound upon being applied inside or onto a wound, comprises water, ozonized oil, glycerol and characterized by astragalas plant extract.

2. A layer according to claim 1, which provides controlled release of active substances by transforming into gel form upon swelling inside or outside the wound.

3. A layer according to claim 1, characterized in that it has a self-adhesive form on the wound.

4. A layer according to claim 1, characterized in that it has a transparent form.

5. A layer according to claim 1, characterized in that it has an elastic form.

6. A layer according to claim 1, characterized in that it has a film form.

7. A layer according to claim 1, characterized by ozonized oil which is ozonized olive oil.

8. A layer according to claim 1, characterized in that it comprises at least one cross-linker for the purpose of increasing mechanical characteristics according to the type of wound where it will be applied and the place of use.

9. A layer according to claim 8, characterized h the cross-linker which is genipin.

10. A layer according to claim 8, characterized by the cross-linker which is calcium chloride.

11. A layer according to claim 1, characterized in that it comprises ozonized oil between 0.5 to 6 (v/v) by percentage in thereof.

12. A layer according to claim 1, characterized in that it comprises glycerol between 2 to 10 (v/v) percentage in thereof.

13. A layer according to claim 1, characterized in that it comprises astragalus plant extract between 0.5 to 3 (w/v) by percentage in thereof.

14. A layer according to claims 8, characterized in that it is obtained by a method of immersion into a solution comprising cross-linker in the ratio of 5 (w/v) by percentage.

15. A layer according to claim 1, characterized by ozonized oil having a peroxide value between the range of 1000 to 1500 meq oxygen/kg oil.

16. A layer according to claim 1, characterized by at least one protection band which covers at least one part thereof and can be during

17. A layer according to claim 1, characterized by at least one adhesive material which is located on the side to be applied to the wound and ensures adherence in the area where it is applied.

18. A layer according to claim 1, characterized in that the part contacting the wound has a hydrophilic structure and the outer part not contacting the wound has a hydrophobic structure.
